(19) United States
(12) Patent Application Publication
(10) Pub. No.: US 2014/0228651 A1
(43) Pub. Date: Aug. 14, 2014

(54) INTEGRATED PORTABLE ANESTHESIA AND SEDATION MONITORING APPARATUS

(71) Applicant: Brainscope Company, Inc., Bethesda, MD (US)

(72) Inventors: Elvir Causevic, San Francisco, CA (US); Robert Hedges, St. Louis, MO (US); Randall Jeffrey Krohn, Wildwood, MO (US); Christian Christiansen, Norresundby (DK); Ralph Walden, Wildwood, MO (US)

(73) Assignee: Brainscope Company, Inc., Bethesda, MD (US)

(21) Appl. No.: 14/256,623

(22) Filed: Apr. 18, 2014

Related U.S. Application Data
(63) Continuation of application No. 11/614,582, filed on Dec. 21, 2006, now abandoned.
(60) Provisional application No. 60/752,357, filed on Dec. 22, 2005.

(51) Int. Cl. A61B 5/00; A61B 5/145; A61B 5/024; A61B 5/0476; A61B 5/0402

(52) U.S. Cl. CPC A61B 5/4064; A61B 5/0476; A61B 5/0402; A61B 5/024; A61B 5/14542
USPC 600/301

(57) ABSTRACT

A system and method for performing monitoring of anesthesia and sedation in a patient includes a patient sensor integrating EEG, pulse oximetry, ECG, and AEP signal inputs, integrated analog hardware, digital hardware, and a digital signal processing system that executes a selected algorithm to process received signals representative of a patient's condition, and which generates an index value associated with said patient condition.

### CROSS-REFERENCE TO RELATED APPLICATIONS

The present application is a non-provisional of U.S. Provisional Patent Application No. 60/752,357 for “Integrated Portable Anesthesia and Sedation Monitoring Apparatus” which was filed on Dec. 21, 2005, from which priority is claimed, and which is herein incorporated by reference.

### STATEMENT REGARDING FEDERALLY SPONSORED RESEARCH

Not Applicable.

### BACKGROUND OF THE INVENTION

The present invention relates generally to a system and apparatus for monitoring levels of anesthesia and sedation in a human or animal patient, and in particular, to an improved monitoring apparatus and system which is self-contained and portable, and which provides an operator with access to a variety of parameters, signal processing algorithms, and a patient database.

In the medical field of anesthesiology, patients must be carefully and continuously monitored to achieve an appropriate balance between delivery of too much or too little of an anesthetic or sedative. Delivery of an inadequate amount of an anesthetic results in a patient being aware of what is happening during a procedure and possible later recall of the procedure, while excessive amounts of the anesthetic or sedative create the risk of damage to the patient's central nervous system from ischemic due to inadequate perfusion. In recent years, the critical importance of depth-of-anesthesia or sedation monitoring has been highlighted by highly publicized incidents of patients' recall of, or sensation awareness during surgery, and incidents of serious injury or death resulting from delivery of excessive amounts of anesthetic. Most anesthesia-related malpractice lawsuits are premised on inadequate monitoring.

The current standards for basic anesthetic monitoring, as specified by the American Society of Anesthesiologists state that “[b]ecause of the rapid changes in patient status during anesthesia, qualified anesthesia personnel shall be continuously present to monitor the patient and provide anesthesia care. During all anesthetics, the patient's oxygenation, ventilation, circulation and temperature shall be continually evaluated.” There is an emerging field of devices that assist the anesthesiologist in monitoring anesthesia, conscious sedation, and deep sedation. This is currently served by passively monitored electroencephalography (EEG) signals.

Similarly, qualified anesthesia personnel are employed to monitor a patient's heart rate and heart condition through electro-cardiogram (ECG) signals, and to monitor a patient's oxygenation through pulse-oximetry readings.

More specifically, known cerebral hemodynamic monitoring techniques include cerebral pulse oximetry and infrared spectroscopy, which measure cerebral oxygen saturation. Transcranial Doppler sonography is a noninvasive technique providing real-time, continuous measurements of blood flow velocity and other hemodynamic parameters such as direction of blood flow and pulsatility in major intracranial vessels. These continuous measurements are utilized as indicators of the status of collateral cerebral circulation, and provide early indications of any disruption of cerebral perfusion which could result in cases of brain ischemia or death.

Electrophysiological monitoring techniques include the use of the electroencephalogram (EEG), such as is described in U.S. Pat. No. 5,287,859 to John, U.S. Pat. No. 6,052,619 to John, and U.S. Pat. No. 6,385,486 to John et al. The degree of randomness of the cortical EEG signal is correlated with the level of awareness of the patient, and is used as an indicator of approaching alertness in a patient. Also, changes in the frequency spectrum, amplitude and phase, statistical properties, coherence, and changes in other measures of the EEG are also used as indicators of changes in the awareness level of the patient. Further, mathematical processing such as wavelet transformation, singular value decomposition (SVD), principal and independent component analyses (PCA/ICA), and other mathematical tools also detect changes in the EEG features that are not detectable using standard techniques, and can provide additional information for accurate gauging of the patient awareness state.

Another known monitoring technique is based on monitoring specific evoked potentials in a selected sensory pathway, such as the auditory pathway. Such a technique is typically employed when certain neural structures in specific sensory pathways are known or believed to be at risk of damage. A sensory stimulus is introduced, and the resulting neural activity generates a wave pattern that is analyzed. The technique relies on adequate discrimination of waveforms using parameters such as peak latency and peak amplitude. Real time changes of the parameters provide a basis for calculating the speed of electrical conduction at the sensory pathway from the peripheral receptor to the sensory cortex. However, evoked signals are intermixed with random EEG activity. To adequately discriminate evoked potentials from random activity, techniques are employed including linear averaging, wavelet processing, statistical analysis and other nonlinear techniques.

The complex auditory evoked potential (AEP) is produced upon presentation of an auditory stimulus or series of stimuli, such as a click or a tone burst, or a complex waveform embedding decoding information for use in later signal processing. The stimuli could be presented to the ear monaurally, with or without masking noise in the contra-lateral ear, or they could be presented binaurally using the same waveform in both ears or different stimulus waveforms to obtain the best signal detection. The AEP consist of early, middle, and late components.

In the early or short latency component of the AEP, the auditory brainstem response (ABR) occurs within 15ms after occurrence of an auditory stimulus and is widely used for clinical evaluation of hearing in infants and other individuals who are unable to effectively communicate as to whether a sound was perceived. In individuals with normal hearing, the ABR generates a characteristic waveform. Auditory testing using the ABR typically involves a visual or statistical comparison of a tested individual's waveform to a normal template waveform. Like other evoked potentials, the ABR is recorded from surface electrodes placed on the patient's scalp. However, the electrodes also record background noise comprised of unwanted bio-potentials resulting from other neural activity, muscle activity, and nonphysiological sources in the environment. The ABR is typically only minimally affected by anesthesia or sedation.

The middle component of the AEP, the auditory mid-latency response (AMLR), also referred to as the middle latency auditory evoked potential (MLAEP) occurs 15 ms-100 ms after occurrence of the auditory stimulus, and is believed to reflect primary, non-cognitive cortical processing of auditory stimuli. Lately, the AMLR or MLAEP has been of particular interest as a measure of the depth of anesthesia.

It is known that the AMLR consists of positive and negative waves that are sensitive to sedatives and anesthetics. In general, increasing the level of sedation or anesthetic increases the latency of these waves, and simultaneously decreases the amplitude. For monitoring purposes, changes in the AMLR waves are quantified as latency to peak, amplitude, and rate of change, and are sometimes combined in a single index.

Alternatively, it is known that a 40 Hz auditory signal can induce an enhanced “steady-state” AEP signal. Conventional signal averaging over a period of time is required to extract the AMLR signal from background EEG signals, but adequate signals usually may be obtainable in about 30-40 seconds. The existence of an intact AMLR is believed to be a highly specific indicator of the awakened state of a patient, and gradual changes in the depth of sedation or anesthesia appear to be reflected by corresponding gradual changes in the AMLR. The AMLR is known to be very susceptible to signal noise.

Another component of the complex AEP, the auditory late response (ALR) is believed to be especially sensitive to the level of sedation or anesthesia applied to a patient, and exhibits a distinct flattening of the waveform at a relatively light level of sedation or anesthesia, among other features. Furthermore, a waveform known as the P300 appears in response to random non-matching stimulus, and is useful for anesthesia monitoring.

The AEPs are characterized as a “weak” bio-signals and present a significant technical problem in analyzing and using the AEP, especially when speed and accuracy are critical. Signal processing techniques using linear averaging, filtering, or conventional denoising are known. However, these techniques remain especially limited in ability to process weak biosignals rapidly and, in some cases, accurately.

Ideally what is needed is a brain activity monitoring technique which is sufficiently sensitive to provide a near instantaneous indicator of small functional changes in a patient's brain. This permits immediate corrective measures to be taken in ample time before patient recall or awareness, or tissue damage occurs. However, known anesthetic monitoring techniques, including those that focus on measures of cerebral perfusion or electrophysiologic function in the brain, are limited in terms of sensitivity and speed, and thus the ability to anticipate and allow timely response to significant functional changes. Against this background, a need exists for improved methods and systems for monitoring the brain function and depth of sedation or anesthesia in a patient.

### BRIEF SUMMARY OF THE INVENTION

Briefly stated, the present invention provides an apparatus and a system which incorporates at least one input representative of a human (or animal) patient's condition, into a self contained, portable, battery powered unit that a practitioner can move between different surgical venues including a hospital's main operating room, outpatient surgery centers, special procedure units, and medical practitioners' offices. One or more inputs are selected from a set of inputs including electroencephalography (EEG), pulse-oximetry monitoring, AEP, breath gas (CO₂) monitoring, and ECG monitoring. The device provides quantitative measures related to a patient's level of consciousness (LOC), in conjunction with optional measurements of blood oxygenation (through pulse oximetry, breath gas monitoring, and ECG). These measures provide information that a practitioner can use, in conjunction with other clinical indicators, to titrate the dose of commonly used anesthetics or sedatives throughout a surgical procedure. The clinical endpoints are patient safety, active management of the level of a patient's consciousness, and the controlled return of the patient to consciousness. These measures can be used individually or combined in a single level of consciousness index to assess overall patient state with respect to anesthesia and sedation administration.

The foregoing features, and advantages of the invention as well as presently preferred embodiments thereof will become more apparent from the reading of the following description in connection with the accompanying drawings.

### BRIEF DESCRIPTION OF THE SEVERAL VIEWS OF THE DRAWINGS

In the accompanying drawings which form part of the specification:

FIG. 1 is an illustration of a self contained, portable, battery powered unit of the anesthesia and sedation monitoring system of the present invention;

FIG. 2 is an illustration of an integrated ECG, EEG, AEP, and pulse-oximetry sensor for use with the system of FIG. 1;

FIG. 3 is a block diagram representation of the interaction between the various hardware components of the system of FIG. 1;

FIG. 4 is a simplified block diagram of the system of FIG. 1, illustrating the interaction of the various components of the system;

FIG. 5 is a block diagram of a software application architecture for the system of FIG. 1;

FIG. 6 is a flow-chart representation of both high-frequency and low-frequency EEG digital signal processing procedures for the system of FIG. 1;

FIG. 7 is a block diagram of a software application architecture for the system; and

FIG. 8 is a block diagram of an EEG analog interface of the present invention.

Corresponding reference numerals indicate corresponding parts throughout the several figures of the drawings. It will be understood that the drawings are for illustrating the concepts of the invention and are not to scale.

### DESCRIPTION OF THE PREFERRED EMBODIMENT

The following detailed description illustrates the invention by way of example and not by way of limitation. The description enables one skilled in the art to make and use the invention, and describes several embodiments, adaptations, variations, alternatives, and uses of the invention, including what is presently believed to be the best mode of carrying out the invention.

The following definitions are used throughout this specification for describing Sedation and Anesthesia according to the American Society of Anesthesiologists (Standards, Guidelines and Statements, 2004):

“Minimal Sedation” (Anxiolysis) is a drug-induced state during which patients respond normally to verbal commands. Although cognitive function and coordination may be impaired, ventilatory and cardiovascular functions are unaffected;

“Moderate Sedation/Analgesia” (Conscious Sedation) is a drug-induced depression of consciousness during which patients respond purposefully to verbal commands, either alone or accompanied by light tactile stimulation. No interventions are required to maintain a patent airway, and spontaneous ventilation is adequate. Cardiovascular function is usually maintained;

“Deep Sedation/Analgesia” is a drug-induced depression of consciousness during which patients cannot be easily aroused but respond purposefully following repeated or painful stimulation. The ability to independently maintain ventilatory function may be impaired. Patients may require assistance in maintaining a patent airway, and spontaneous ventilation may be inadequate. Cardiovascular function is usually maintained; and

“General Anesthesia” is a drug-induced loss of consciousness during which patients are not arousable, even by painful stimulation. The ability to independently maintain ventilatory function is often impaired. Patients often require assistance in maintaining a patent airway, and positive pressure ventilation may be required because of depressed spontaneous ventilation or drug-induced depression of neuromuscular function. Cardiovascular function may be impaired.

Because sedation is a continuum, it is not always possible to predict how an individual patient will respond. Hence, practitioners intending to produce a given level of sedation should be able to rescue patients whose level of sedation becomes deeper than initially intended. Individuals administering Moderate Sedation/Analgesia (Conscious Sedation) should be able to rescue patients who enter a state of Deep Sedation/Analgesia, while those administering Deep Sedation/Analgesia should be able to rescue patients who enter a state of general anesthesia.

A condition known as burst suppression sometimes occurs during the administration of anesthesia and sedation. It is characterized by a specific EEG waveform containing bursts of EEG activity followed by suppression of EEG activity in subsequent time periods. This condition is indicative of a patient's awareness level, generally corresponding to deeper states of anesthesia.

The anesthesia and sedation monitoring system of the present invention, indicated generally 10 in the drawings, provides a level of consciousness (LOC) index of a patient P to a practitioner administering anesthetic agents to the patient during a surgical procedure. The LOC index indicates, such as on a scale of 0-99, the level of the patient's brain activity, so to guide the administration of the agents. Optional secondary functions provide a measure of the level of oxygenation in the patient's system via a pulse oximetry patient interface which consists of a finger or forehead sensor and associated cable, and a measure of the patient's breath gases such as CO₂ via a cannula drawing breath gases to a capnometer. The LOC index may integrally incorporate information from any of the above specified measures, or the individual measurements may be presented as stand-alone indices. Preferably, the monitoring system 10 is a highly portable instrument, which is pole or bench mounted, and which preferably provides a display having a good distance visibility for the various clinical indices.

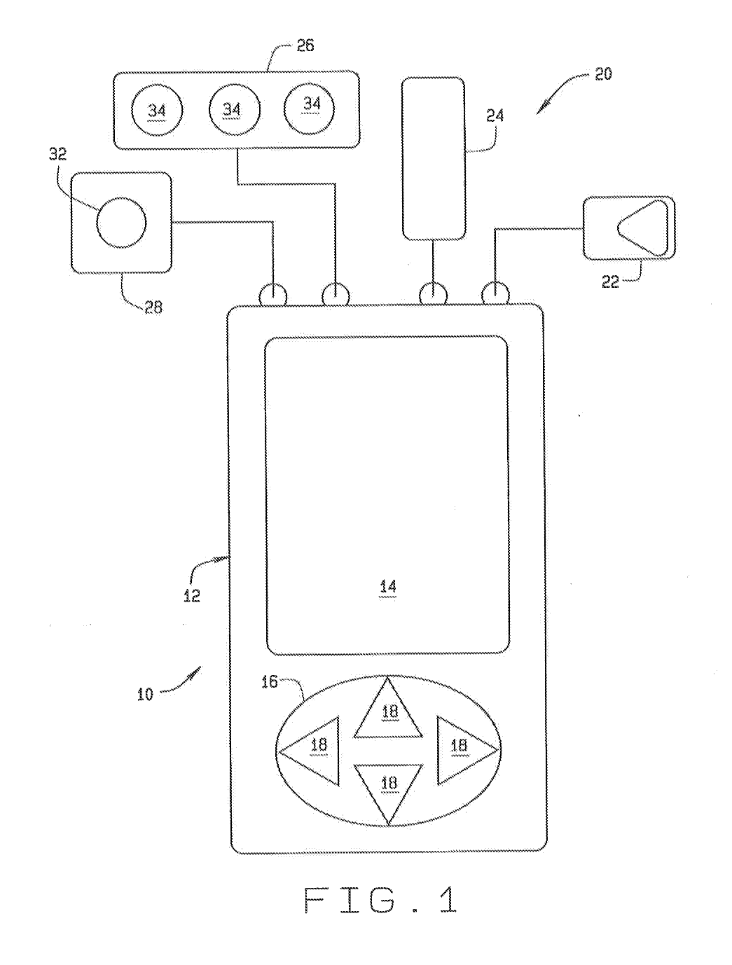

An embodiment of anesthesia and sedation monitoring system 10, and as shown in FIG. 1, includes a self contained, portable, battery powered unit or device 12 that a practitioner can move between different surgical venues within a hospital. These include operating rooms, outpatient surgery centers, units where special procedure are performed, and the offices of medical practitioners.

The unit 12 has a display 14 which includes a color display, preferably with touch sensitivity, and a control section 16 comprising a keypad having buttons or keys 18 by which a user can use the display. Unit 12 also contains analog and digital hardware with appropriate internal couplings to other functional blocks in order to implement the desired functionality of the anesthesia and sedation monitoring system 10.

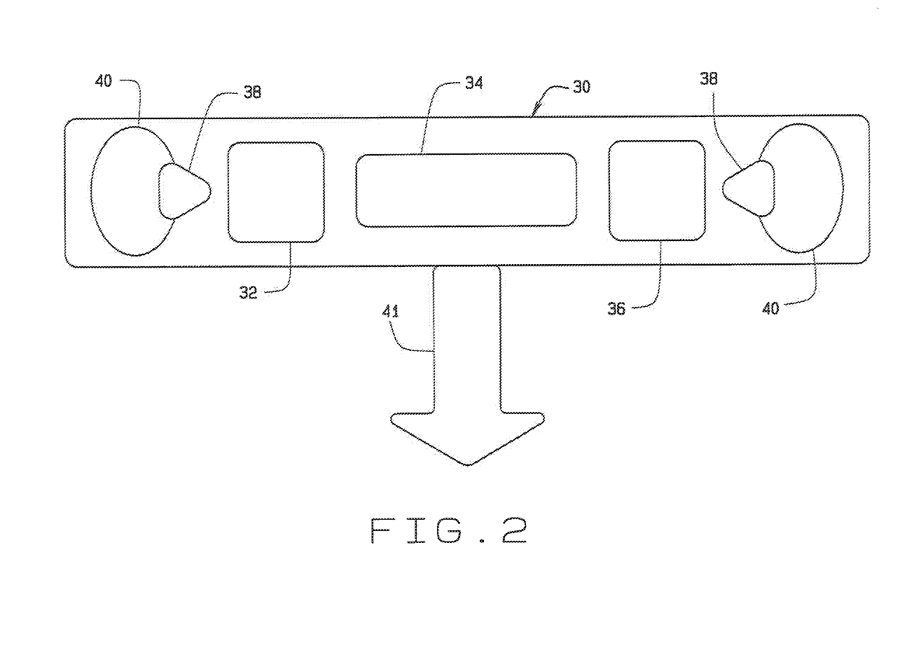

A sensor suite 20 includes various sensors one or more of which are connected to unit 12 at any one time to provide patient information to medical personnel. Four such sensors are shown in FIG. 1 and include an AEP transducer 22, a pulse-oximeter (OX) sensor 24, an EEG sensor 26, and an ECG sensor 28. Operation of such sensors is known in the art and is not described. As shown in FIG. 2, sensors 22-28 are incorporated into a headband 30. The headband includes an electrode 32 for ECG sensor 28, three (3) electrodes 34 for EEG sensor 26, a sensor element 36 for pulse-OX sensor 24, and an AEP transducer 38 for AEP transducer 22. The headband may further includes ear openings 40 at each end of the band for a patient to conveniently wear the band across the forehead with the various electrodes and sensors positioned against the scalp, such as at the temple region. The respective outputs of sensor suite 20 are routed from headband 30 to unit 12 via a connector 41 having a double row connector plug (not shown) which attaches to a receptacle (also not shown) on unit 12. Signals from electrodes 32, 34 are preferably routed through a front end preamplifier 35.

An alternative embodiment of system 10 is constructed of unit 12, a Patient Interface Cable 201, and electrode array 26. The Patient interface cable (PIC) 201 is primarily constructed with an Instrumentation Amplifier (IA) 142, and a gain and filter stage 144. This signal path provides the input to the Analog to Digital Converter (ADC) 146. This signal path may take the form of a single-ended or differential analog interface.

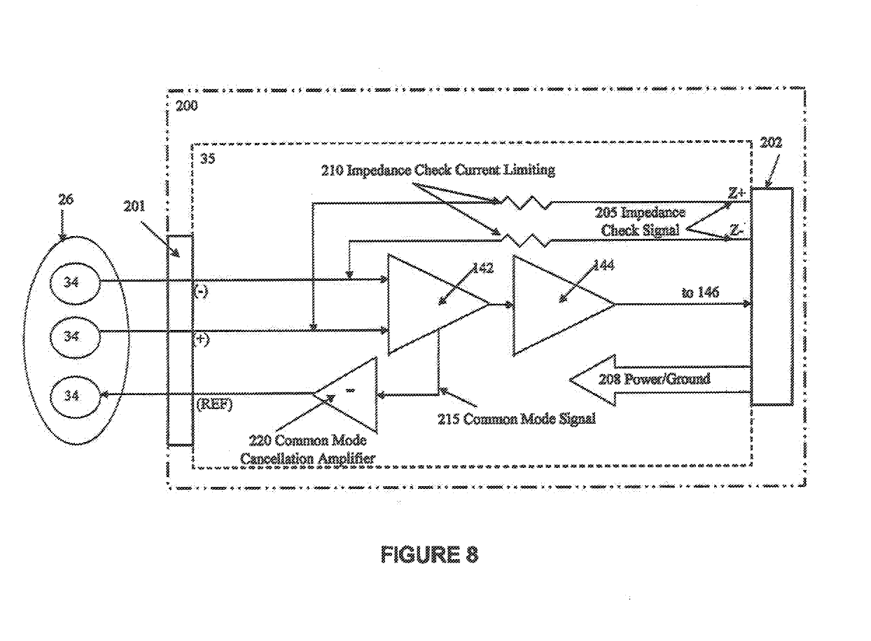

The Patient interface cable (PIC) 201, such as shown in FIG. 8, may additionally embody a common-mode cancellation amplifier 220 that filters and inverts the common-mode signal 215 that is derived from the Instrumentation Amplifier 142. This common-mode cancellation amplifier configuration serves to attenuate the amount of common-mode signal that might otherwise appear in the signal digitized by ADC 146. The common-mode cancellation amplifier has a specific frequency response designed to operate on signals within a specified frequency band. Common-mode cancellation amplifier 220 is configured such that the effective output impedance is very low, with patient auxiliary current limiting provided within the feedback loop of an inverting amplifier. This low effective output impedance further serves to reduce common-mode noise that would otherwise be impressed upon the signal due to differences in alternating current potential between the patient and the monitoring device. These potential differences occur because the patient, the device, and other patient connected equipment each have varying degrees of capacitive coupling to alternating current mains and earth ground.

The Patient interface cable (PIC) 201 may additionally embody an impedance check circuit composed of impedance check input signal 205 and impedance check current limiting impedances 210. Synchronous application of signal 205, digitizing with 146, and analyzing with a SNAP module 60 will allow for an estimation of impedances formed by electrodes 34 and the patient's skin.

The Patient interface cable (PIC) 201 may additionally embody an electrode tracking system to be used to verify that the connected electrode is an authentic OEM product, and to automatically detect the specific type or functionality of the electrode system being used (i.e. adult vs. pediatric, with or without ECG feature, with or without AEP feature, etc.).

Requisite signals for the operation of the patient interface cable (PIC) 210—including Power and Ground 208, Impedance Check Signals 205, and the amplifier EEG signal (applied to ADC 146) are all routed through a shielded cable 202, which in turn connects to unit 12 through a connector (not shown).

Alternately, patient interface cable (PIC) 201 may embody all the requisite amplification, filtering and ADC conversion, such that the signals out of the patient interface cable (PIC) are fully digitized. This alternative embodiment may incorporate an analog-to-digital converter 146 within the patient interface cable (PIC) 210 to transmit a digitized version of the amplified EEG signal to Finite Impulse Response (FIR) filter and decimate processing unit 148 through the shielded cable 202.

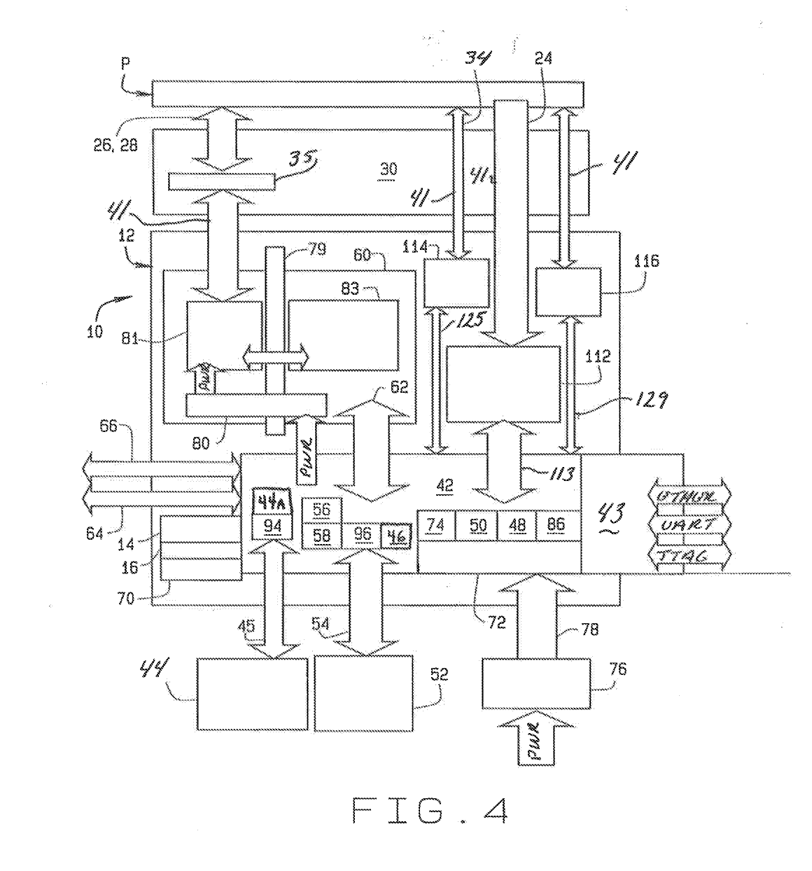

The processor system 42, see FIG. 4, preferably comprises a Texas Instruments (TI) OMAP 5910 dual core processor including an ARM-9 core and a TI 320C55X digital signal processor (DSP) core. The two processors feature an integrated means of communication and data sharing which facilitates cooperative operation of the two processors. Permanent program storage is implemented in a FLASH memory 44A which is a non-volatile storage medium that facilitates easy changes of programming. An expansion module 43 can be interfaced with the processor system 42 if desirable. The expansion module may provide network connectivity, such as to an Ethernet, as well as Universal Asynchronous Receiver Transmitter (UART) and JTAG capabilities. Those of ordinary skill in the art will recognize that the processors and hardware components described herein may be altered, replaced, or supplemented, without departing from the scope of the invention, with different processors or hardware components having sufficient computational capacity to carry out the operational functions of the anesthesia and sedation monitoring system described herein.

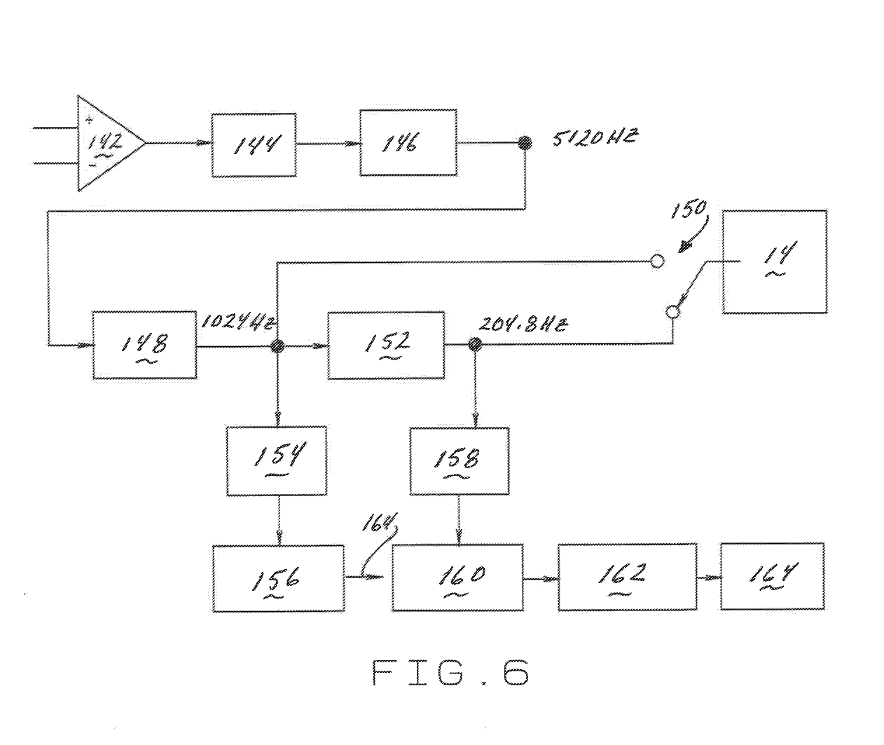

During operation of the system 10, data from the respective sensors in sensor suite 20 is first collected in a sample buffer 48 until the buffer is filled, at which time an interrupt signal is generated to stop further data collection until the accumulated data is processed. Alternately, the collected data may be continually streamed for processing at the processor system 42. During processing, the digital signal processor (DSP) 42 moves the data from the sample buffer 48 to a working buffer 50. The digital signal processor (DSP) may employ a window function, a data saturation function, and other time-domain integrity checks. If the received data is acceptable, an LOC calculation is performed to generate the LOC index value. Preferably, the LOC algorithm includes a Fast-Fourier Transform (FFT) and various filter functions. Alternately, the LOC algorithm includes additional mathematical tools, linear and non-linear, such as wavelet processing, SVD, PCA/ICA, etc. As is shown in FIG. 6 and discussed hereinafter, additional processing is then performed for separate frequency bands in the FFT output. The LOC algorithm is periodically executed and involves use of overlapping input data vectors. Results from a series of computations are periodically averaged together to produce an output value. In one embodiment the slow rate of the output of the LOC allows the processor system 42 to communicate to a host Personal Computer (PC) 52 which communicates with unit 12 through a universal serial bus (USB) 54 in real-time. The averaged result is then stored in a shared memory 56 and an interrupt from the digital signal processor (DSP) is issued to the processor. From shared memory 56, the data is moved to a memory section 58 of processor 42 for storage and display.

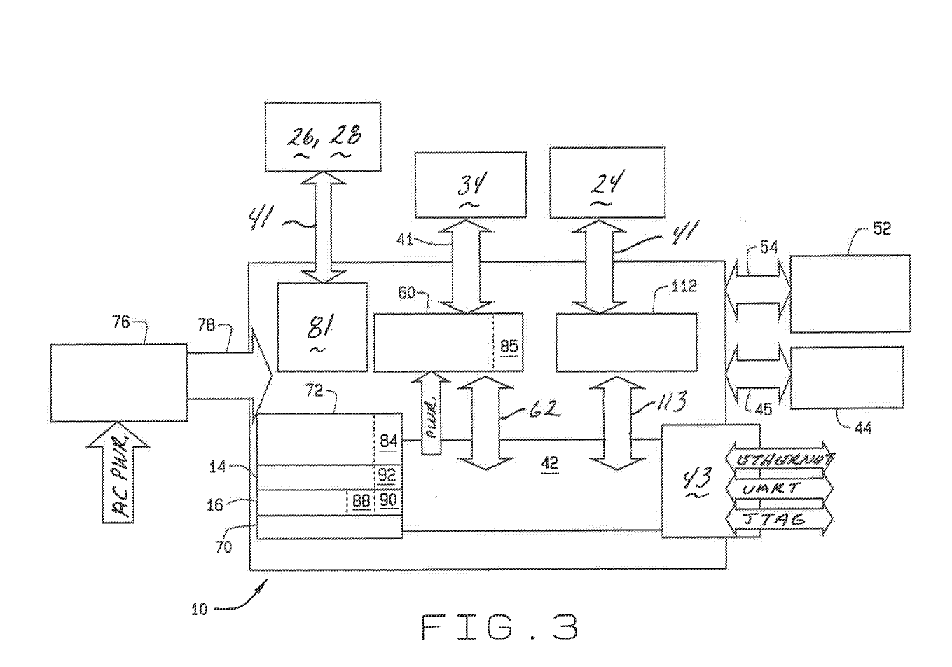

Preferably, in one embodiment of the present invention, data collection functions are handled by a SNAP module 60 connected to unit 12, and which contributes to patient safety electrical isolation per IEC 60601-1-1 and 60601-2-26 requirements. A proprietary communication bus 62 is utilized to transfer data between SNAP module 60 and the OMAP processor system 42. As shown in FIG. 3, the SNAP module 60 is self-contained and is configured in such a way that it may be interfaced with standard multi-parameter monitors as a removable module; as well as with portable anesthesia and sedation monitoring system 10 of the present invention.

The primary function of the SNAP module 60 is to implement the LOC algorithm. This algorithm processes the acquired EEG waveforms and provides an indication of the patient's LOC. Other information may be incorporated into the LOC, such as the AEP, EKG, CO₂, etc. The calculation to provide this information is data driven. A control CPU 46 initializes the digital signal processor (DSP) using an Application Program Interface (API) function and then uses another API function to commence data collection. In an alternate embodiment the LOC algorithm may be implemented directly on the digital signal processor (DSP) of the processor system 42.

The SNAP module 60 may additionally embody an electrode tracking system to be used to verify that the connected electrode is authentic, and to automatically detect the type of electrode system used (i.e. adult vs. pediatric, with or without ECG feature, with or without AEP feature, etc.).

Patient data records, when optionally recorded, may be stored on external FLASH memory cards 44 using a Compact Rash (CF) card format. Anesthesia and sedation monitoring system 10 will operate with or without a CF card 44 inserted; however, no patient record storage will be available if a CF card or other storage media is not provided. This includes storage of raw EEG/AEP data.

The primary user interface to system 10 is touch interface 14. The touch interface is used to initiate tests, manage record storage and retrieval, and control and respond to alarms. An audible alarm is incorporated in the Printed Circuit Board (PCB) for keypad 16. The secondary user interface to the device is keypad 16. A “standby” state of the device is changed by pressing a standby power button (not shown). Other buttons (also not shown) are used to perform specific functions as defined in the software specification for system 10.

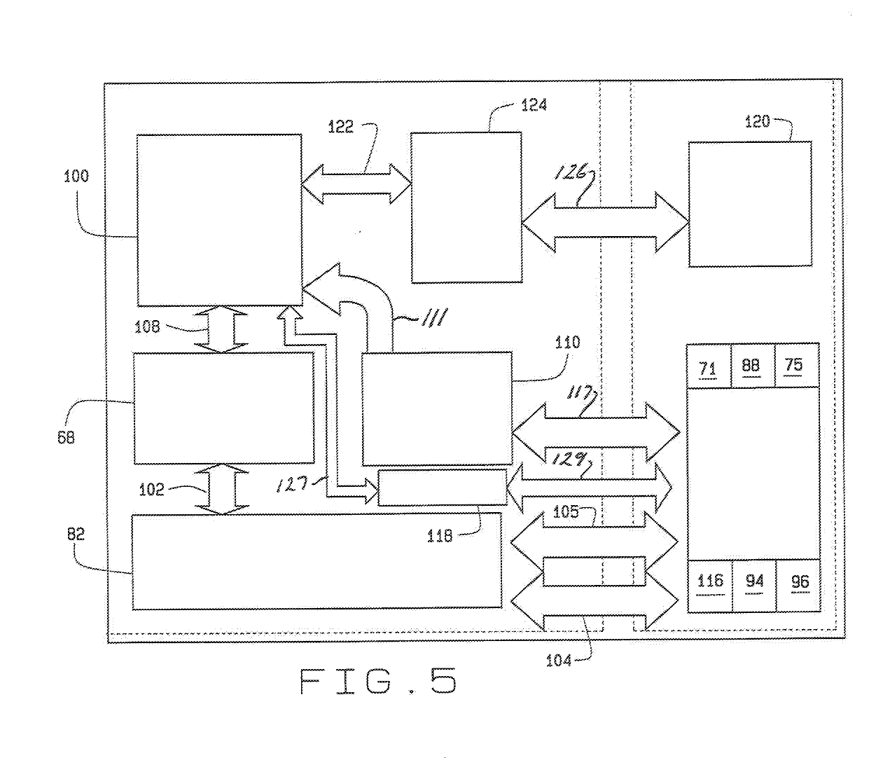

A display 14 provides the main feedback to a user regarding the current operating state of device 12 and the LOC of the patient. A Graphical User Interface (GUI) 68, see FIG. 5, is employed to visually depict the operating state of the unit, and the state of patient P, in a consistent manner. Color LEDs indicators 70 preferably indicate the charging (Standby) state and operating (ON) state of the unit.

Preferably, anesthesia and sedation monitoring system 10 of the present invention is battery operated using an internal battery pack 72 which includes, for example, one or more Lithium ion batteries. Current to charge the battery is supplied by a UL recognized medical-grade power supply 76. A battery management module 74 of OMAP processor 42 monitors the charge/discharge cycles of the batteries. Battery charge monitoring does not utilize any software; rather, charge current to battery 70 is provided by power supply 76 through an appropriate charging connection 78. A dedicated non-rechargeable battery (not shown) provides power to a real-time-clock (also not shown) in unit 10 when the device is not operating, thus maintaining the correct time when the unit is in its “standby” mode.

The anesthesia and sedation monitoring system 10 incorporates various electrical isolation barriers in the internal design. In the preferred embodiment, unit 12 includes a 4mm creepage gap in the printed circuit board (PCB) components installed in the unit, and employs opto-isolators 79 where components are required to bridge the gap between PCBs of a front end SNAP module 81 and a SNAP data module 83 for data transfer between the modules. An isolated power source 80 includes a DC-DC converter and provides 1 kVDC isolation and 5 VDC to the isolated portions of SNAP module 60 as shown in FIG. 4. To avoid any interference with the bandwidth of the various input signals, a switching frequency of converter modules is selected to be outside the desired signal spectrums, and to greatly exceed the system signal sampling rates.

The operating system (OS) for the anesthesia and sedation monitoring system 10 invention is preferably an embedded version of a Linux® operating system or any other commercially available or custom operating system, so to provide the necessary elements for a multi-threaded application suitable for the purposes of the invention. The OS enables programming through readily available tools such as a Qt GUI Library 82, see FIG. 5, and C programming language. Specific tasks are assigned to their own threads, which the OS schedules as resources become available. Alternatively, other types of a graphical or non graphical user interface may be utilized. The availability of a multi-threaded OS facilitates the development of separate programming threads to handle patient LOC computations, GUI operation, alarms, and communications, among others. In addition, the Linux OS employs driver modules to help perform various tasks or functions with external hardware or device subsystems. These include a battery monitor driver 71, an alarm driver 88, a driver 75 for display 14, a driver 99 for CF card 44, and drivers 96 for the USB systems.

A SNAP module driver 85 performs the interfacing required for the OMAP Processor platform to work with the SNAP module hardware. The driver relays commands and data through a defined interface to affect control and communication for the module.

A battery monitor driver 84 interacts with a battery monitor circuit to monitor the charge status of battery pack 72. The battery monitor circuit uses a 1-wire or HDQ serial port to affect communications. Processor system 42 also has an integrated 1-wire or built in HDQ communications controller and an associated driver 86. Driver 86 performs all required setup and interpretation of the bit stream from the communication controller, and reports the data through a structure defined by a main program for unit 12.

Alarm driver 88 controls supply of power to an acoustic transducer 90 on a PCB for keypad 16 in response to a command from the main program. The driver manages the port and controls hardware I/O for the transducer. A touch screen driver 92, CF card driver 94, and a USB driver 96 regulate power and data flows to these components.

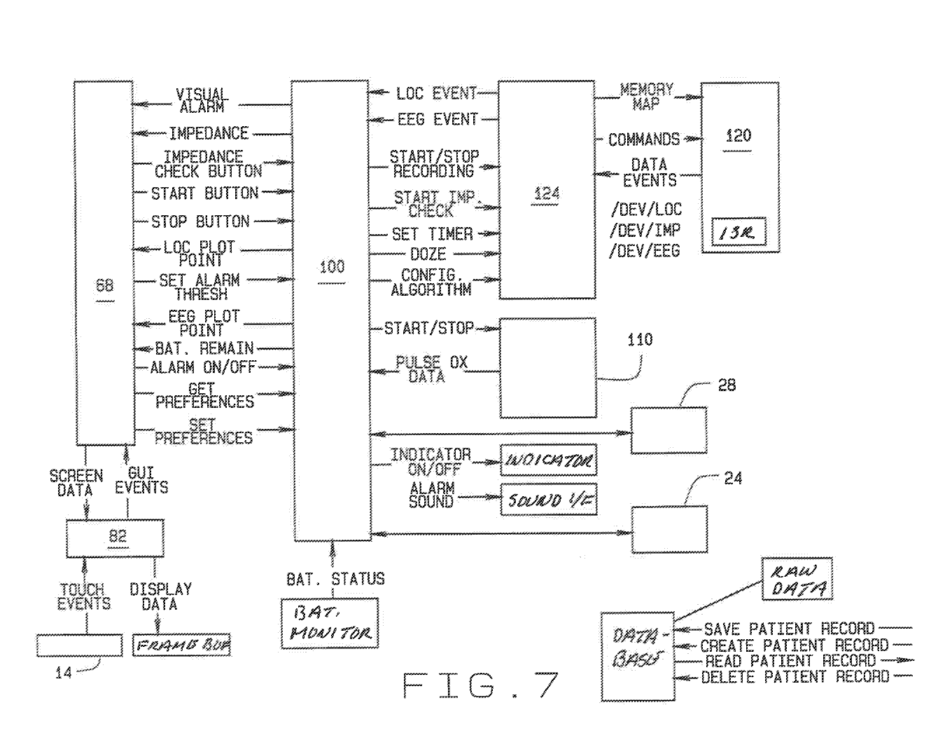

A main application code module 100 for system 10 is shown in FIG. 5 and the code is used to perform all setups and initializations steps, configurations and starts-ups. Once this is accomplished, the code establishes a loop through a message queue which is in an infinite loop. The software architecture implemented in system is shown in FIG. 7 and includes the various information transmitted between the different modules or threads.

GUI 68 uses a Qt development system from Trolltech. A single path 102 interfaces the GUI with Qt library 82. A path 104 allows the GUI to manage touch screen and a path 106 allows the GUI to manage a LCD frame buffer for display 14. Both paths allow bi-directional communications through Qt library 82. The GUI communicates with main application code module 100 through a bi-directional GUI message queue 108.

Next, main application code module 100 interfaces with a LOC module 120 through a bi-directional LOC message queue 122, a LOC library module 124, and a communications path 126 between modules 120 and 124, to implement the command structure and the data structure.

The battery management function periodically generates requests for battery charge status through the battery monitor circuit. Returned data is used to drive a graphical battery gauge (not shown) on the GUI.

The pulse oximetry function includes a serial port driver 110 which handles communications with a pulse oximetry module 112 (see FIG. 4). Driver 110 communicates with module 100 through a two-way path 111. Module 112 communicates with processor system 42 through a UART path 113 and driver 110 through a path 117. Driver 110 is a standard Linux kernel mode driver which is configured at the time of kernel compilation. Module 100 further communicates with an ECG/AEP module 116 using an ECG driver 118 with which module 110 communicates over a path 127. Driver 118 communicates with module 116 over a path 129.

During operation, system 10 is configured to provide audible alarms, using an alarm system 114 (see FIG. 4), in response to one or more events, some of which may occur simultaneously. Communications between processor system 42 and alarm system 114 are via a communications path 125. The events include the occurrence of an LOC index value exceeding upper or lower threshold levels optionally established, as well as standard pulse oximetry alarms, such as SpO2 sensing interruption, SpO₂ low, heart rate high, and heart rate low. System 10 is further configured to identify various error conditions which may occur based on periodic or continuous measurements. These error conditions include, for example, inappropriate electrode impedance, low quality EEG signal, device functional error conditions, low battery conditions with an estimate of remaining battery life, and dead battery conditions. Similar conditions are monitored and detected for ECG and capnometry as well.

To facilitate the storage of patient information, system 10 preferably generates a database record for each procedure performed on each patient, when the patient database option is enabled through an optional memory card. The procedure database preferably contains basic patient information (in compliance with local healthcare facility policies regarding HIPPA). This information may include a patient ID, patient name, gender, birth date, weight, height, allergies, and associated notes. Additional information stored in the database may include clinician information (identifying anesthesiologists, surgeons, and other attending staff, an index trend (including AEP), SpO₂ tred, heart rate, and end-tidal CO/CO₂ concentrations.

In addition to the procedure database, a second, optional comprehensive database contains processed, but undecimated measurement data streams including: continuous EEG waveform samples; SpO₂ Pleth waveform (when available); ECG waveform (when available); and CO/CO₂ waveforms (when available).

In the preferred embodiment the above data streams can be temporarily stored in a circular buffer in the memory of the processor system 42. The number of procedures that may be stored in system 10 depends upon the durations of procedures, and the size of available memory storage; and, may be flexible. Preferably, an external PC viewing program is available for printing information, and to allow practitioners to log their procedures into the system for documentation purposes.

In addition to the databases, system 10 may also be configured to store all input keystrokes and operational states in compliance with HIPPA guidelines, for purposes of problem operating issues reported by users.

To facilitate storage and exchange of data, a device communication interface is provided in the system of the present invention. Preferably, this interface includes at least one CF 44 memory expansion port through which data is transferred to either an external device or an external memory, or the interface allows updating of application software running on the system. Those skilled in the art will recognize that other device communication interfaces may be provided, including, but not limited to, MIB (IEEE 1073 Medical Information Bus) compliant interfaces, USB, IRDA, Ethernet (TCP/IP), RS-232, Bluetooth or other wireless link, or other removable storage interface (Memory Stick, etc.)

To acquire bioelectric signal data from a patient, system 10 employs a single-use non-sterile electrode array. The array preferably comprises the three electrodes 34 which form one differential EEG channel with a reference. The electrodes 34 are physically connected to each other as part of EEG sensor 26, but are electrically isolated and terminated with a standard 5-pin positive latch connector designed to mate with patient interface cable 41. The patient's skin could be prepared by wiping it with an alcohol pad, for example. As is conventional, electrodes 34 are constructed with conductive gels specifically designed for use with EEG electrodes. As previously described, the array is part of a headband 30 worn by the patient for the duration of a procedure.

During use, the electrodes 34 are preferably placed on the forehead of the patient. The preferred practice is for one input electrode (+) or (−) to be placed on the centerline of the forehead, the other input electrode to be placed above the temple, and the middle to be placed over the eye. The electrodes may be placed on either side of the forehead.

Part or all of the integrated sensor suite 20 may be embedded in headband 30.

The preferred embodiment of the sensor suite 20 and headband 30 could be made disposable or reusable depending upon manufacturing cost, device reuse requirements, and cleaning requirements. An alternative permutation would have portions of the sensor suite 20 disposable with other portions of sensor suite 20 being reusable, with headband 30 being reusable. The integrated sensor suite may be battery operated or supplied with power by the anesthesia system.

Integrated sensor suite may additionally embody an electrode tracking system to communicate to the anesthesia system that the connected electrode is authentic, and to automatically inform the anesthesia system of the type of electrode system that is being connected (e.g. adult vs. pediatric, with or without ECG feature, with or without AEP feature, etc.).

The present invention is implemented, in part, by computer-implemented processes and apparatus for performing those processes. The present invention can also be embodied, in part, in the form of computer program code containing instructions embodied in tangible media, such as floppy diskettes, CD-ROMs, hard drives, or other computer readable storage media. In this regard, when computer program code is loaded into, and executed by, an electronic device such as a computer, micro-processor or logic circuit, the device becomes an apparatus for practicing the invention,

Referring to FIG. 6, processor system 42 further processes an output from a FFT in the LOC algorithm for separate frequencies in the output frequency band of the signal. As shown in FIG. 6, the EEG sensor signal output is supplied to an Instrumentation Amplifier 142 whose output is directed to an anti-aliasing filter unit 144. The analog output from anti-aliasing filter 144 is provided to a 16-bit Analog-to-Digital Converter (ADC) 146. The output of the ADC is a high bandwidth (e.g., 5120 Hz) signal having both a mid-range and low range frequency component. This output from the ADC is supplied to a Finite Impulse Response (FIR) filter and decimate processing unit 148. The filter and decimate processing unit checks the data stream for artifacts, filters the data with an appropriate decimation FIR filter and decimates the data to provide a mid-range component signal. The mid-range (e.g., 1024 Hz) component of the signal from FIR 148 is routed to a switch 150 for display on display 14 as a real-time EEG display.

The mid-range frequency component of the signal from FIR 148 is directed to both a FIR filter and decimate unit 152 and a sample and storage module 154. Data in module 154 is processed using a FFT and High Frequency (HF) protocol as indicated at 156. The result of this processing is directed to a SNAP index 164.

If an artifact is detected in the filter and decimate processing unit 148, a quick restart algorithm is used to refill the sample and storage module 154. In the preferred embodiment the sample and hold module contains 10s of contiguous samples in 10 1 s buffers. To reduce the lag required to refill these buffers after an artifact event, a quick restart algorithm fills the buffers with manipulated copies of the 1 s buffer of data acquired after the artifact event. The buffers are numbered sequentially 0-9 with buffer 0 corresponding to the buffer to be filled with data from the current acquisition and buffer 9 corresponding to data acquired 9 seconds previous. The quick start algorithm fills buffer 0 and the other even-numbered buffers with the currently acquired data, whereas the odd-numbered buffers are filled with a time-reversed versions of the buffer. This alternating pattern of forward-reversed data provides 10 s of data while eliminating jump discontinuities across buffers. By eliminating the jump discontinuities, we reduce the resulting spurious high frequency components which would be present in the frequency domain analysis that follows in module 158. Those skilled in the art will recognize that the specific parameters of the quick restart algorithm can be adjusted to optimize performance, and that a different combination of buffers could be used to accomplish the same task.

The output from FIR 152 is a low-range (e.g., 204.8 Hz) component of the signal and is supplied to display 14 through switch 150. The output from FIR 152 is also directed to a sample and storage module 158. Data in module 158 is processed using a FFT and Low Frequency (LF) protocol as indicated at 160. The result of this processing is directed to SNAP index 164 through a burst suppression module 162. Burst suppression module detects the presence of the burst suppression patient condition in the EEG and incorporates that information into the index. Alternately, burst suppression module informs the user, via a GUI, that the patient has entered burst suppression state, and may indicate to the user the parameters of the burst suppression waveform—such ratio of burst to suppression, % burst, etc.

In one embodiment the above SNAP index could be further augmented by including information from ECG, AEP, CO/CO_{—}2 and SpO2 sensors in the index calculation.

Finally, the present invention can be embodied, in part, in the form of computer program code, for example, whether stored in a storage medium, loaded into and/or executed by a computer, or transmitted over some transmission medium such as electrical wiring or cabling, through fiber optics, or via electromagnetic radiation. When computer program code is loaded into and executed by a computer in this way, the computer becomes an apparatus for practicing the invention. When implemented in a general-purpose microprocessor, the computer program code segments configure the microprocessor to create specific logic circuits. This includes an embodiment that performs all the specified functions in a stand-alone module, OEM module, without user interface, which could be operatively coupled to any standard multiparameter patient monitor (Philips, GE, Siemens, etc.).

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1-45. (canceled)

46. A device for determining a brain state of a patient, the device comprising:
a headset comprising a plurality of different biosensors configured to acquiring a plurality of different bioelectric signals from the patient; and
a base unit operatively connected to the plurality of different biosensors on the headset through a signal acquisition interface cable;
wherein the base unit comprises a signal processor configured to process the acquired bioelectric signals from the plurality of different biosensors and to utilize data corresponding to the plurality of different bioelectric signals to generate at least one index value indicative of the brain state of the patient,

47. The device of claim 46, wherein the plurality of different biosensors include an EEG sensor, an ECG sensor, and an auditory transducer.

48. The device of claim 46, wherein the plurality of different bioelectric signals include an EEG signal, an ECG signal, and an auditory evoked potential response signal.

49. The device of claim 46, wherein the headset further comprises a pulse-oximeter sensor configured to measure at least a pulse and an oxygenation level of the patient.

50. The device of claim 49, wherein the signal processor is further configured to process the pulse and the oxygenation level of the patient and to integrate data corresponding to the pulse and the oxygenation level in generating the at least one index value indicative of the brain state of the patient.

51. The device of claim 46, wherein the headset further comprises a breath gas analyzer configured to receive the patient's breath gases and to measure at least a level of CO₂ in the received breath gases.

52. The device of claim 51, wherein the signal processor is further configured to process information related to the patient's breath gases including the level of CO₂ and to integrate data corresponding to the patient's breath gases in generating the at least one index value indicative of the brain state of the patient.

53. The device of claim 46, wherein the base unit comprises a display device configured to provide a visual display of the at least one index value indicative of the brain state of the patient.

54. The device of claim 46, wherein the base unit further comprises a memory unit.

55. The device of claim 54, wherein the memory unit stores a population database comprising bioelectric signals from a plurality of individuals.

56. The device of claim 46, wherein the index value calculated by the processor is indicative of the level of sedation or anesthesia of the patient.

57. The device of claim 46, wherein the signal acquisition interface cable comprises at least one of an amplification circuit, a signal filtering circuit, and an analog-to-digital conversion circuit.

58. The device of claim 46, wherein the signal acquisition interface cable comprises an impedance check circuit configured to provide an impedance check signal to the headset.

59. The device of claim 46, wherein the impedance check circuit is configured to enable automated checking of the impedance at an interface between the patient's skin and an electrode of one of the biosensors.

60. The device of claim 46, wherein the headset further comprises a tracking system configured to communicate to the base unit the types of biosensors that are connected to the patient.
